(19) United States
(12) Patent Application Publication
(10) Pub. No.: US 2015/0305623 A1
(43) Pub. Date: Oct. 29, 2015

(54) METHODS FOR LOCALIZING AND TREATING A PARATHYROID ADENOMA

(71) Applicant: Invuity, Inc., San Francisco, CA (US)

(72) Inventors: James Bredenkamp, Mission Viejo, CA (US); Alex Vayser, Mission Viejo, CA (US)

(73) Assignee: Invuity, Inc., San Francisco, CA (US)

(21) Appl. No.: 14/694,895

(22) Filed: Apr. 23, 2015

Related U.S. Application Data
(60) Provisional application No. 61/983,068, filed on Apr. 23, 2014.
(60) Provisional application No. 61/983,910, filed on Apr. 24, 2014.
(60) Provisional application No. 62/003,986, filed on May 28, 2014.

(51) Int. Cl. A61B 5/00; A61B 18/04; A61K 49/00

(52) U.S. Cl. CPC A61B 5/0066; A61K 49/0017; A61B 5/0075; A61B 18/04; A61B 5/0071; A61B 2018/046; A61B 2018/00577

(57) ABSTRACT

Parathyroid adenomas may be identified using optical probes which distinguish the diseased tissue from adjacent healthy tissue. Spectroscopic properties of the tissue may be used to distinguish healthy from diseased tissue. Traditional surgery to remove the parathyroid adenoma may be avoided by using techniques to occlude the blood supply to the diseased tissue. Occlusion may be accomplished by applying energy to the blood vessel such as with radiofrequency energy, ultrasound energy or laser energy. Cryogenic probes may also be used to occlude the blood flow. Clips, sutures or other mechanical mechanisms may also be used to occlude the flow, thereby destroying the diseased tissue.

### CROSS-REFERENCE

The present application is a non-provisional of, and claims the benefit of U.S. Provisional Patent Application Nos. 61/983,068 (Attorney Docket No. 40556-734.101) filed Apr. 23, 2014; 61/983,910 (Attorney Docket No. 40556-734.102) filed Apr. 24, 2014; and 62/003,986 (Attorney Docket No. 40556-734.103) filed May 28, 2014; the entire contents of which are incorporated herein by reference.

### BACKGROUND OF THE INVENTION

Primary hyperparathyroidism (HPT) is a common disease that affects 1 in 500 women and 1 in 2000 men. It is characterized by hypercalcemia and elevated parathyroid hormone (PTH) levels. It mainly affects persons in their middle years of life, with a peak incidence between 50 and 60 years of age. Uncontrolled HPT is characterized by a multitude of symptoms including kidney stones, osteoporosis, hypertension, peptic ulcer disease, cardiovascular disease, muscle weakness, bone pain, depression and decreased intellectual processing. Most often it is caused by a single benign tumor, called a parathyroid adenoma, which develops on one of the typically four parathyroid glands in the neck. Surgery (parathyrodectomy) is the undisputed treatment with removal of the affected parathyroid gland and the adenoma.

Parathyroid surgery is technically challenging primarily because it is often difficult to localize intra-operatively the diseased gland. Pre-operative localization is therefore crucial to help limit surgical dissection, reduce operative time, decrease complications, and eliminate surgical failure. It is the key component in the decision to perform an outpatient parathyrodectomy. The literature contains considerable controversy surrounding which test or tests are best to localize the diseased parathyroid gland. In the past, parathyroid adenomas have been localized by a modality of methods including nuclear medicine, ultrasound, MRI and CT scanning, fine needle aspiration, and selective venous sampling of PTH. At this time, the most commonly utilized studies are the sestamibi SPEC nuclear medicine exam and ultrasound. Most patients are now are getting both studies at considerably high cost. Preferably, an ideal pre-operative localization test would be inexpensive, highly sensitive, specific, and minimally invasive. No pre-operative localizing test currently fits this description. Moreover, it would be also be advantageous to provide methods and devices that allow a surgeon to treat the parathydroid adenoma more accurately and more efficiently than traditional surgical removal. At least some of these objectives will be satisfied by the devices and methods disclosed herein.

### BRIEF SUMMARY OF THE INVENTION

In one aspect of the present invention, a method for localizing a parathyroid adenoma in a patient comprises inserting a probe into the patient, optically scanning tissue with the probe, and distinguishing the parathyroid adenoma from adjacent healthy tissue based on the optical scan.

Inserting the probe may comprise percutaneously inserting the probe into the patient. Optically scanning the tissue may comprise spectroscopically scanning the tissue. The method may further comprise disposing a fluorescent dye in the patient. Optically scanning may comprise scanning the tissue with optical coherence tomography. The method may further comprise ischemically ablating the distinguished parathyroid adenoma. Ischemically ablating may comprise occluding blood flow to the distinguished parathyroid adenoma by applying radiofrequency energy, ultrasound energy, a chemical agent, a mechanical occlusion device, or applying a cryogenically cooled probe to a blood vessel supplying the blood flow.

In another aspect of the present invention, a method for treating a parathyroid adenoma in a patient comprises restricting blood flow to a parathyroid adenoma, and ischmically ablating the parathyroid adenoma.

Restricting blood flow may comprise occluding the blood flow by applying radiofrequency energy, ultrasound energy, a chemical agent, a mechanical occlusion device, or applying a cryogenically cooled probe to a blood vessel supplying the blood flow.

In another aspect of the present invention, a device for localizing and treating a parathyroid adenoma in a patient comprises an optical probe configured for identifying the parathyroid adenoma from adjacent healthy tissue.

The optical probe may comprise a spectroscopic probe. The device may further comprise an ischemic ablation element for occluding a blood vessel supplying blood to the parathyroid adenoma. The ischemic ablation element may comprise a radiofrequency electrode, an ultrasound transducer, a cryogenic probe, a chemical agent, an occlusion clip, or a mechanical occlusion element.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic diagram of the posterior view of the thyroid showing the parathyroid glands and their blood supply.

FIG. 2 illustrates the blood supply to a parathyroid.

FIG. 3 illustrates a probe sensing diseased tissue adjacent healthy tissue.

FIG. 4 illustrates a flowchart of an exemplary method of detecting and treating diseased tissue.

FIG. 5 illustrates an exemplary probe for detecting diseased tissue and for providing a therapy to the diseased tissue.

FIG. 6 illustrates a spectroscopic curve for various tissues.

FIG. 7 illustrates reflected light off of various tissues across the entire wavelength range.

### DETAILED DESCRIPTION

Specific embodiments of the disclosed device, system, and method will now be described with reference to the drawings. Nothing in this detailed description is intended to imply that any particular component, feature, or step is essential to the invention.

Most surgeons and many endocrinologists are now skilled in the technique of ultrasound. It has been shown to have a sensitivity of 85% and a specificity of 94%. Ultrasound is limited in that it can only identify abnormal appearing tissue, and cannot differentiate between lymph node, thymus, or ectopic thyroid. For these reasons, it is often combined with sestamibi to provide information about the functional status of the tissue. Needle aspiration of the mass can also assist in identification, but requires additional processing, costs, and time. We describe a novel new optical method which provides the accuracy of ultrasound with the specificity of fine needle aspiration. It has the benefit of instant operator feedback without the need of complex pathological processing and costs. It is inexpensive and can be done in the office setting by both surgeons and endocrinologists. Additionally, methods for treating the parathyroid adenoma are also disclosed herein.

FIG. 1 is a schematic diagram of the posterior view of the thyroid showing the parathyroid glands and their blood supply.

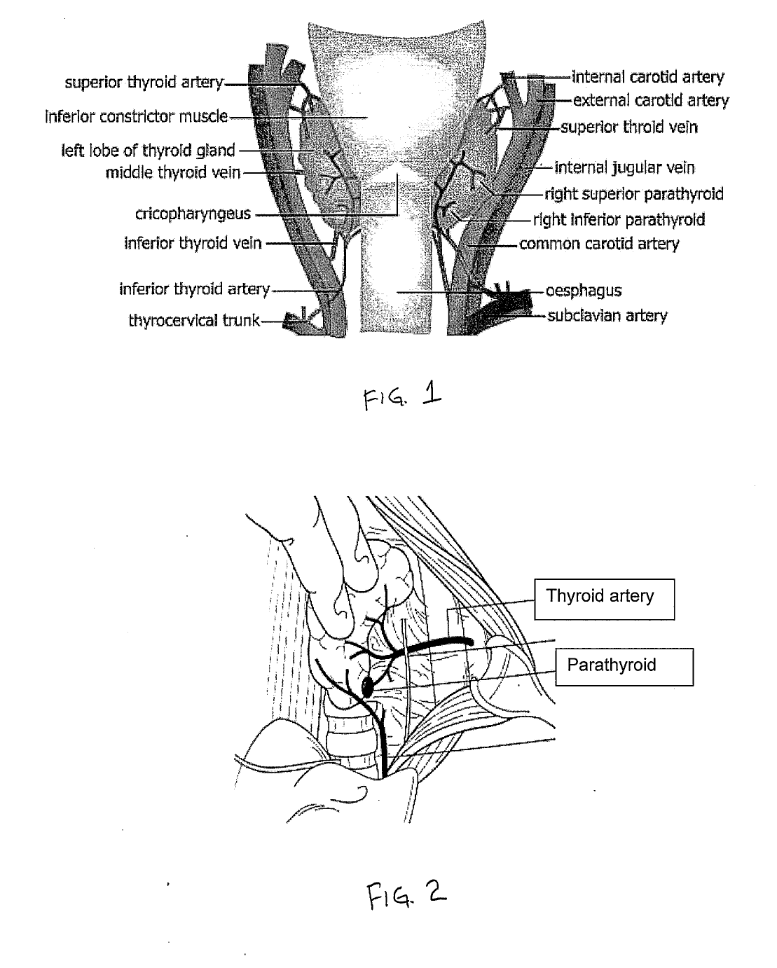

FIG. 2 illustrates the blood supply to a parathyroid.

### Localization

When a patient presents with biochemical evidence of primary hyperparathyroidism (elevated serum calcium, elevated PTH, normal Vitamin D, and normal to elevated urine calcium), the surgeon or endocrinologist skilled in diagnostic ultrasound can utilize an optical probe to percutaneously confirm that a structure seen on ultrasound and suspicious for being a parathyroid adenoma is in fact a parathyroid adenoma. Specifically, in the office as an outpatient, an optical probe is passed through the skin and a quantifiable reading is made of a tissue mass. Depending on the true identity of the mass, the optical probe would tell the operator if the mass in question was thyroid tissue, parathyroid tissue, fat, thymus, or a lymph node. If the mass was identified as parathyroid tissue, then the surgeon would know exactly where to go at the time of surgery to find the parathyroid adenoma, and the patient would then proceed directly to surgery without the need for additional and costly testing. This would obviate the need for fine needle aspiration with additional time and complex costly processing of the tissue, and in most situations also avoid costly and time consuming nuclear scanning with Sestamibi or CT/MRI scan.

FIG. 3 shows a probe 34, preferably an optical probe disposed through the patient's skin and sensing diseased tissue 32 such as parathyroid tissue adjacent healthy tissue T such as thyroid tissue. The probe may be any of the probes disclosed in this specification.

Various optical methods can be used to perform this test. The optimum method is done by spectroscopy. If a known tissue such as parathyroid has different reflective spectroscopic properties compared to adjacent tissue, such as thyroid, lymph nodes or fat, the optical probe would be localized by ultrasound over suspected tissue and would identify what that tissue is by its spectroscopic signature. Fluorescence can also be used to identify the tissue. It is known in the art that parathyroid auto-fluoresces. That is when excited by a certain wavelength, it will emit a different wavelength that is different that adjacent tissue. Fluorescence can also be performed with a dye such as IC Green (Indocyanine green). As this dye is injected vascular structures will “light up” when exposed to excitation wavelengths of IC Green. The probe thus can detect this as well. Lastly, more advanced optical methods such as (OCT) Optical Coherence Tomography can be used to identify the tissue. Using this well-known technique, one can use the probe to see the image of the parathyroid vs. adjacent tissue. This could be used in conjunction with another modality such as spectroscopic probe.

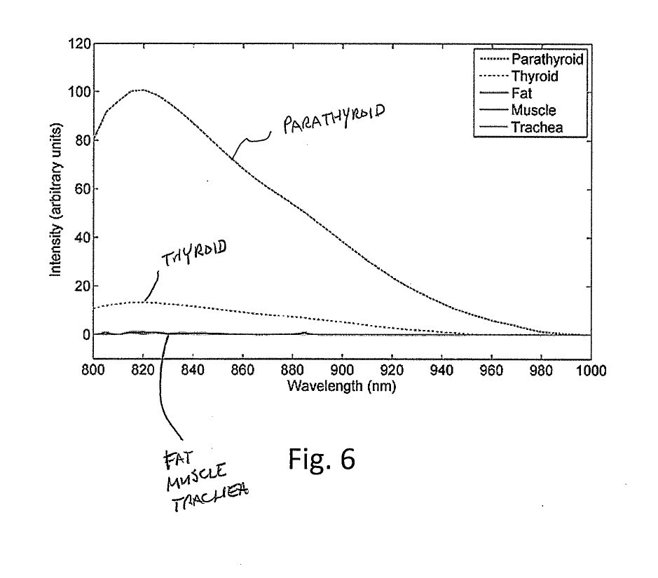

Other research has demonstrated that the parathyroid exhibits different fluorescence intensity than adjacent tissue, such as disclosed in US Patent Publication No. 2012/0010483 which used Raman spectroscopy; the entire contents of which are incorporated herein by reference. FIG. 6 shows an exemplary curve for various tissues. The disclosure in US 2012/0010483 was conducted using an open procedure with direct contact between the probe and the tissue.

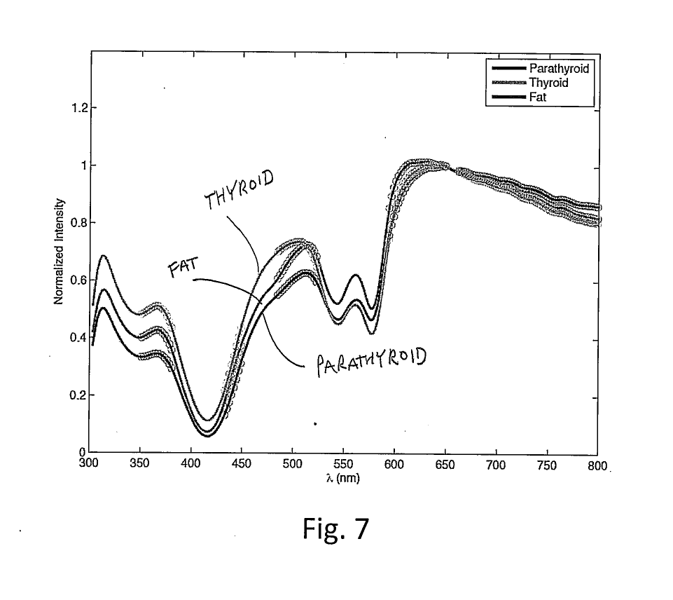

Elastic Scattering Spectroscopy may also be used to help distinguish the diseased tissue from the healthy tissue. FIG. 7 illustrates reflected light off of various tissues across the entire wavelength range.

Both techniques demonstrated that parathyroid tissue has different optical properties vs adjacent tissues. There are other methods that have been described in the literature such as optoacoustic probes which may use infrared light presented on the tissue though a fiber and an acoustic signal is then received by an acoustic transducer. Various tissues, will exhibit different acoustic signatures and can be differentiated.

Additionally, various peptide or aptamer probes can be designed to target specific tissues. Aptamer probes for example are small DNA or RNA molecules that are known to have capacity to recognize specific epitopes or antigens, and with affinities close to that of antibodies. Once designed and selected, they are stable and can be injected into the patients and paint the specific tissue of interest with a fluorescent marker. The fluorescence can then be imaged with the fiber probe/camera for localizing the parathyroid.

Lastly a different type of a probe can be used to identify the tissue. It is well known that tissue and bone can conduct electrical current. Various tissue will conduct at different rates. Thus, similar to spectroscopic approach and conductive approach can be taken to differentiate tissue.

### Treatment

The primary treatment patients who develop primary hyperparathyroidism is surgery. Aside from being invasive and with traditional surgical risks, there is also a financial downside to this treatment due to the cost associated with operating room time, cost of consumables, etc. In the past, various percutaneous methods have been evaluated to treat hypercalcemia associated with primary hyperthyroidism for patients who are not surgical candidates (patients with single remaining parathyroid gland). This approach was with ultrasound guided ethanol ablation.

A key procedure step during this approach was to localize and identify the parathyroid tissue. This was done with fine needle aspiration or the sestamibi test. As described above this has been traditionally accomplished with fine needle aspiration or the sestamibi test. This may be eliminated by using the proposed spectroscopic probe described above.

With ethanol, the needle was typically inserted directly into the parathyroid tissue under the ultrasound guidance and ethanol was injected. There have been other approaches using heat/laser energy. However, most of these approaches have had similar results to the use of ethanol targeted to the adenoma. All of which did not have successful long term effects as the PTH and calcium levels in patients increased over time.

The proposed new method uses a different approach where the blood supply is cut off thereby causing ischemia to the parathyroid, sometimes also referred to as ischemic ablation. As seen from FIGS. 1 and 2 each of the parathyroid glands has a vascular supply that is delivered through the thyroid artery. By using energy or some other means, the vessel supplying blood to the tissue may be coagulated or otherwise closed thereby destroying the parathyroid adenoma tissue. Thus blood vessel supplying blood to the tissue may be shut by using energy such as radiofrequency energy, ultrasound energy including high intensity focused ultrasound (HIFU), microwave or laser energy. Cryogenic techniques may also be used to close the blood supply. Also, chemical treatments or mechanical methods such as using a microclip or a suture may be used to shut the blood supply.

### Experiment 1

Based on early results, we have clipped off the thyroid artery that fed into an inferior parathyroid. Prior to clipping, the patient's PTH was at 159 pg/ml. After clipping we tested for PTH in two time intervals (5 and 10 min) At 5 min the PTH dropped to 55.3 pg/ml and at 10 min dropped to 29.9 pg/ml. Further long term clinical trial is needed to understand the long term effects of this approach, however stopping blood supply to the parathyroid appears to be promising technique for treating the adenoma.

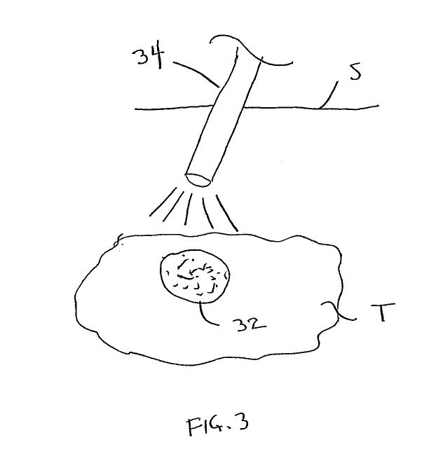

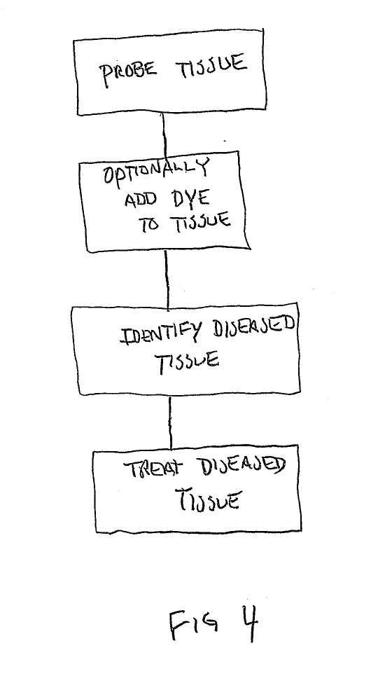

FIG. 4 illustrates a flowchart outlining the exemplary method of detecting the diseased tissue and treating the diseased tissue.

A preferred embodiment of the parathyroid treatment device integrates these modalities into a single tool. Therefore, the surgical instrument may comprise an optical probe that can detect the parathyroid tissue and differentiate from others. This tool can have additional modalities so to localize the parathyroid and ablate it using one of the technologies described above. It is also possible to provide a micro cannula, where the cannula fits over the localizing probe. Once the parathyroid is identified, the cannula is left in place and probe is pulled out. Another probe for treating the tissue (such as the RF probe, cryo probe, etc. described above) can be placed inside the cannula to provide the therapeutic function.

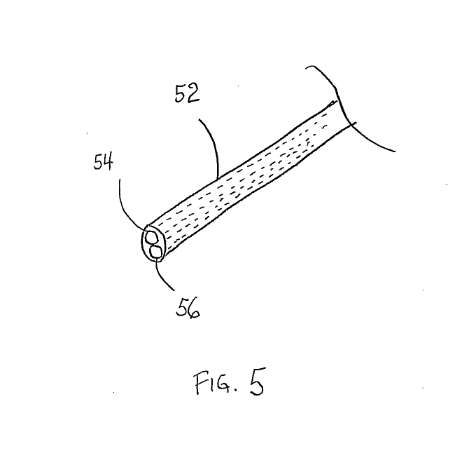

FIG. 5 illustrates an exemplary embodiment of a combined detection and treatment probe 52. The probe 52 includes a tissue probe 54, preferably an optical probe such as those described herein or any of the other tissue identification probes disclosed herein which can identify the diseased tissue. The probe 52 also includes an ablation treatment element 56 which may deliver any of the treatment therapies described above. A single probe allows identification of the tissue to be treated and allows treatment to be provided with a single device.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for localizing a parathyroid adenoma in a patient, said method comprising:
inserting a probe into the patient;
optically scanning tissue with the probe; and
distinguishing the parathyroid adenoma from adjacent healthy tissue based on the optical scan.

2. The method of claim 1, wherein inserting the probe comprises percutaneously inserting the probe into the patient.

3. The method of claim 1, wherein optically scanning the tissue comprises spectroscopically scanning the tissue.

4. The method of claim 1, further comprising disposing a fluorescent dye in the patient.

5. The method of claim 1, wherein optically scanning comprises scanning the tissue with optical coherence tomography.

6. The method of claim 1, further comprising ischemically ablating the distinguished parathyroid adenoma.

7. The method of claim 1, wherein ischemically ablating comprises occluding blood flow to the distinguished parathyroid adenoma by applying radiofrequency energy, ultrasound energy, a chemical agent, a mechanical occlusion device, or applying a cryogenically cooled probe to a blood vessel supplying the blood flow.

8. A method for treating a parathyroid adenoma in a patient, said method comprising:
restricting blood flow to a parathyroid adenoma; and
ischemically ablating the parathyroid adenoma.

9. The method of claim 8, wherein restricting blood flow comprises occluding the blood flow by applying radiofrequency energy, ultrasound energy, a chemical agent, a mechanical occlusion device, or applying a cryogenically cooled probe to a blood vessel supplying the blood flow.

10. A device for localizing and treating a parathyroid adenoma in a patient, said device comprising:
an optical probe configured for identifying the parathyroid adenoma from adjacent healthy tissue.

11. The device of claim 10, wherein the optical probe comprises a spectroscopic probe.

12. The device of claim 10, further comprising an ischemic ablation element for occluding a blood vessel supplying blood to the parathyroid adenoma.

13. The device of claim 12, wherein the ischemic ablation element comprises a radiofrequency electrode, an ultrasound transducer, a cryogenic probe, a chemical agent, an occlusion clip, or a mechanical occlusion element.
